(19) United States
(12) Patent Application Publication
(10) Pub. No.: US 2017/0049978 A1
(43) Pub. Date: Feb. 23, 2017

(54) RESUSCITATION ARRANGEMENT COMPRISING MASK, MONITORING ARRANGEMENTS, AND DIGITAL MODULE DETACHABLY ARRANGED AS PART OF THE MASK

(71) Applicant: Monivent AB, Göteborg (SE)

(72) Inventors: Mattias BERG, Göteborg (SE); Maria LINDQVIST, Göteborg (SE); Oscar EDVARDSSON, Göteborg (SE); Pontus JOHANSSON, Göteborg (SE); Jesper LINDGREN, Göteborg (SE); Kenneth SANDBERG, Sävedalen (SE); Mikael LÖFGREN, Mölndal (SE); Ants SILBERBERG, Vallda (SE)

(21) Appl. No.: 15/307,653

(22) PCT Filed: Apr. 17, 2015
(86) PCT No.: PCT/SE2015/050445

§ 371 Date: Oct. 28, 2016

(30) Foreign Application Priority Data
Apr. 30, 2014 (SE) 1450517-6

(51) Int. Cl. A61M 16/00; A61B 5/00; A61M 16/06; A61B 5/091

(52) U.S. Cl. CPC A61M 16/0003; A61B 5/091; A61B 5/6803; A61M 16/06; A61M 16/0078; A61M 16/0084; A61B 2503/04; A61B 2560/0443; A61M 2240/00; A61M 2016/003; A61M 2205/15; A61M 2016/0027; A61M 2205/587; A61M 2205/60; A61M 2205/3584; A61M 2205/8206; A61M 2205/52; A61M 2205/502

(57) ABSTRACT

A resuscitation arrangement and method for resuscitation of humans, preferably infants, wherein said arrangement for resuscitation comprises a mask adapted to fit over the nose and mouth of said human, monitoring arrangements adapted to monitor at least one parameter chosen from flow, volume, leakage, and pressure within said mask and a digital module adapted to enable measurement of at least one of the parameters. The monitoring arrangement comprises at least one passive sensor component and an active sensor component, wherein the digital module is adapted to be detachably arranged as a part of said mask and the passive sensor component is arranged within the mask and the active sensor component is arranged in the digital module.

### TECHNICAL FIELD

The present invention relates generally to a resuscitation arrangement for resuscitation of humans, preferably infants.

### BACKGROUND ART

It is an ever so often occurring situation that an infant during birth requires assistance because of difficulties to start breathing. This occurs especially when there are complications with the birth process independent if the situation is that the child is delivered prematurely or at term. One of the most common situations is that the child is in need of immediate assistance with breathing which normally is done by blowing or pumping air in to the child. Resuscitation is also common in many other cases and can be conducted on any human independent of his or her age.

In prior art it is known to provide arrangement for resuscitation of humans that are manually controlled by a care provider, such as doctor or a nurse. The care provider adjust the amount of gas that passes in to a human being resuscitated manually through for example a manual ventilation means or through actuating a valve attached to a gas source. The manual ventilation means can for example be a manual pump and the valve can be any form of valve that allows the care provider to adjust the air flow. The art of resuscitation of humans thereby requires special skills of the care provider providing the treatment which are difficult to acquire. The situation where live training can be conducted is in general life threatening situations for the patient and the only means to determine success or failure are by observing the patient. This procedure, and especially the observation of the patient have proven to be especially difficult if the patient is an infant or small child.

The method of resuscitation generally comprises that the care provider utilizes a mask adapted to fit over the nose and mouth of the patient. The care provider then manually adjusts the amount of gas, preferably oxygen, that is transferred through the mask and into the human.

At hospitals there are currently no devices that provide assistance to the care provider in those situations and as previously stated most of the adjustments are done based on the care provider's experience. The care provider normally assesses the situation from criteria such as if the color of the child improves, if the heart rate is increasing, or if the chest is moving. This leaves many parameters unknown such as if the child gets enough oxygen, is the volume sufficient but not too large, etc. Those parameters are all important for a successful result and it should for example be noted that too small amounts of gas or air would create a dangerously low oxygen level while a level exceeding the required amount might damage the lungs.

The prior art shows some solutions that shows the pressure of the air pumped in to the human. This provides some guidance but also provides a false security, for example if the human has a problem in the throat and no air can pass through, a pressure is still built up no or insufficient amounts reaches the lungs. Furthermore, even if the pressure is within the right interval there is no guarantee that the provided volume of gas is sufficient.

In prior art, furthermore a few solutions providing additional functionality are available that mainly are adapted for training purposes and can provide information to the care provider about certain values relevant to the success or failure of the treatment process. However, such systems in general have plenty of drawbacks such as increased dead space, uncertainties in relation to the accuracy of the information as well as drawbacks in presentation of the information. Furthermore, all such solutions comes with additional problems such as tubes or wires connecting the sensors to displays or any other type of devices displaying the information to the care provider. In terms of live situations this means that the care provider normally has to rely only on his or her experience because such monitoring device normally is not available or operational.

It would thereby be beneficial to provide a solution for monitoring the resuscitation process without increasing the dead space in a resuscitation mask, without decreasing the maneuverability and mobility of the resuscitation arrangement as well as provide improved means for indication of success or failure.

### SUMMARY

An object is to provide an improved resuscitation arrangement that comprises means to monitor crucial parameters without substantially increasing the dead space. Another object of the present invention is to improve the feedback for resuscitation arrangement and thereby also improve the patient security.

Thus, the solution relates to a resuscitation arrangement for resuscitation of humans, preferably infants, wherein said arrangement for resuscitation comprises a mask adapted to fit over the nose and mouth of said human, monitoring arrangements adapted to monitor at least one parameter chosen from flow, volume, leakage, and pressure within said mask and a digital module adapted to enable measurement of at least one of the parameters. The monitoring arrangement comprises at least one passive sensor component and an active sensor component, wherein the digital module is adapted to be detachably arranged as a part of said mask and the passive sensor component is arranged within the mask and the active sensor component is arranged in the digital module.

The resuscitation arrangement can be any form of arrangement adapted for resuscitation of a human and arranged in any one of multiple different locations such as a hospital, an ambulance, or any other care providing facility such as a maternity ward. Depending on the application area and conditions at the location where the resuscitation arrangement is arranged the resuscitation arrangement can have different components and designs.

In one embodiment the resuscitation arrangement is a stationary arrangement arranged in a hospital wherein the resuscitation arrangement comprises a main unit connected to the power grid and optionally also connected to a network, over Ethernet, WiFi, Bluetooth, or any other form of wired or wireless network. The resuscitation arrangement further comprises a mask connected to the digital module. The mask can be any form of mask suitable to fit over the nose and mouth of a human and it is understood that the mask can have many different sizes and shapes as well as being formed from different materials. Furthermore, the resuscitation arrangement comprises means to supply gas to the patient. The gas can be any form of gas suitable for the purpose but preferably normal air, oxygen, or compressed oxygen. Depending on the setting the oxygen can be provided from a gas tube or directly from the air. In one embodiment the gas is supplied by a hand through a manual resuscitator in another the gas is supplied by a gas tube connected to a manual valve or a T-piece resuscitator. In yet another embodiment the supply of gas is totally manual.

The resuscitation arrangement can further comprise additional components known to the person skilled in the art, such as a PEEP (positive end-expiratory pressure) valve.

The resuscitation arrangement can further be adapted to measure different parameters such as the volume entering the mask, the volume coming back from the lungs, the difference in-between the latter two in order to establish leakage, ventilation rate, compliance or resistance from the lungs, and oxygen saturation.

Pressure can in one embodiment be monitored as PIP (Peak inspiratory pressure) indicating the highest total pressure and PEEP (positive end-expiratory pressure) the remaining pressure in the lungs.

By arranging the digital module detachably as part of the mask as previously described many advantages can be achieved. For example, in one embodiment the mask is a mask that is intended to be used multiple times and it is thereby a requirement that the mask can be washed between usages. At the same time, solutions comprising tubes, or any other means adapted to transport gas, in order to conduct measurements comprises unnecessary amounts of dead space which is one objective to avoid. In order to avoid dead space sensors should optimally be arranged within the mask itself but such an arrangement would create problems when washing the mask. Sensors that would be washed over and over again would both be exposed to unbeneficial environments during the washing sequence but also require calibration more often. By arranging the digital module detachably as a part of the mask those problems are avoided while the dead space is kept to a minimum. Yet another benefit is that the decreased amount of tubes improves the usability and flexibility of the resuscitation arrangement.

The active sensor component can be any form of electrical sensor component, such as a pressure sensor, flow sensor, gas sensor, volume sensor, or any other form of sensor. The passive sensor component is a component of the sensor which does not comprise any electrical parts and thereby is substantially mechanical in its nature. In one preferred embodiment the passive sensor part is a constriction limiting the flow area in a flow path of the resuscitation arrangement.

In one embodiment of the resuscitation arrangement the digital module is adapted to be detachably arranged in a recess of the mask.

There are many advantages with arranging the digital module within a recess of the mask except from being an esthetically appealing arrangement. The mask is in one preferred embodiment held in the hand of the operator while pressed downwards towards the mouth and nose of the patient. This means that it is beneficial if the mask fits easy within the hand of the operator and thereby it is advantageous if the digital module can blend in with the general design of the mask as much as possible. This is achieved by a digital module arranged in a recess of a mask. Furthermore, the amount of dead space both within the piping leading to and from the mask as well as in the mask itself should be kept to a minimum. The dead space affects the accuracy in measurement and if increased it actually thereby increase the risk suffered by the patient being treated. By arranging the digital module within a recess in the mask the digital module occupies space that for most masks of the prior art otherwise would be dead space.

Arranging the digital module within a recess of the mask furthermore minimizes the risk of accidentally detaching any tubes or sensor cables since the sensor connections could be hidden within the recess and thereby out of access for the operator during normal operation.

In one embodiment of the resuscitation arrangement the digital module is detachably arranged as part of said mask through a magnetic engagement.

The digital module may be attached with different engagement means such as magnetic or mechanical. Mechanical can for example be a clip of any sort or a tight fit between the mask and the digital module. In one embodiment, magnetic engagement can be used as further described below.

In one embodiment of the resuscitation arrangement the digital module is a reusable module and the mask is a disposable mask.

In different embodiments the mask can be either a disposable or a reusable mask. A reusable mask could for example be a washable mask that can be used multiple times as described above together with some advantages provided for reusable masks. The solution also provides advantages for disposable masks that are used for one or a few times only before disposed of. The disposable mask is in one embodiment preferred due to the contamination of the mask after usage. In comparison to the mask itself the sensor components, and especially the active sensor components, are significantly more expensive which would make a disposable mask comprising such components too expensive for most application areas. This problem is solved through the detachable digital module while the dead space, as previously disclosed, is kept to a minimum.

In one embodiment of the resuscitation arrangement the passive sensor component comprises a first and second sensor outlet adapted to be in fluid connection with the digital module, and wherein said passive sensor component comprises an arrangement creating a pressure difference between said first and second sensor outlet.

There are multiple ways of measuring pressure that can be applied. For example, tubes be utilized that are inserts into the flow channel in the form of pressure outlets. The differential pressure can thereby be measured directly in the flow. Another solution is to utilize mechanical means to determine the flow, such as a rotating wheel, or any other form of mechanical means that can determine a flow in real time. Another solution that is used in a preferred embodiment is to introduce a constriction in to the flow path working as flow impedance. This causes the pressure to drop on each side of the impedance which relates to the flow and thereby can be used to determine the flow. The passive sensor component is in one preferred embodiment such a constriction. It is further understood that the person skilled in the art could arrange any form of sensor, active sensor component, and passive sensor component.

In one embodiment of the resuscitation arrangement the digital module further comprises an illumination arrangement adapted to illuminate the mask.

Due to the limited availability of monitoring equipment for resuscitation processes medical doctors operating in the field relies on their ability to monitor the progress by analyzing vital signs of patient. In doing so the medical doctor normally focuses their attention to the patient and it has shown inconvenient to display information about the progress solely on a display located anywhere else than in the close vicinity of the patient. However, arranging a display in the close vicinity of the patient presents multiple problems and also provides the risk of distraction from other important tasks if the treatment method for example suddenly changes. This means that it would be beneficial to provide indication means on the mask itself which has proven to be a challenging task due to the way the mask, especially the mask for small children and infants, are held. Large parts of the mask are during operation often covered by the operator's hands. This has created a solution where indication lights and similar has been ineffective to use. By providing means to illuminate the entire mask a more effective means for indication is achieved.

In one embodiment of the resuscitation arrangement the illumination arrangement comprises at least one light guiding material, preferably fiber optics or light guiding plastic.

The illumination arrangement could be any form of illumination arrangement that can illuminate said mask. This is in one embodiment achieved through arranging fiber optics within the mask that illuminates. In another embodiment, a light guiding material is used within the mask making the mask itself supporting the illumination. The illumination can for example be powered by one or more LED (light emitting diode) arranged in the digital module.

In one embodiment of the resuscitation arrangement said illumination arrangement is adapted to shine in different colors depending on the parameters measured by the sensors. The colors could in one preferred embodiment be red in the case that any problem, such as significant leakage, has occurred or green if the parameters are within predetermined values.

In one embodiment of the resuscitation arrangement said illumination arrangement is adapted to pulsate or blink.

In one embodiment it is beneficial if the illumination arrangement makes the mask blink in order to indicate to the operator the current status. By creating an illumination arrangement that blinks instead of being illuminated all the time power can be saved increasing the battery life of the digital module.

In one embodiment of the resuscitation arrangement the mask comprises an identification arrangement enabling for said digital module to identify at least one specification of said mask.

The person skilled in the art understands that the size of mask required for resuscitation might vary between for example a small child and a grown human. The size of the mask is a parameter that affects the results for the crucial parameters that are measured. This means that it would be beneficial for the digital module to know what kind of mask it is currently attached to which can be achieved through the arrangement in accordance with the present solution. The arrangement for enabling the digital module to identify at least one specification of the mask can for example be an arrangement that identifies the size of the mask. In one embodiment this is achieved by micro-switches arranged on the bottom surface of the digital module that are engaging with corresponding arrangements in the mask. The corresponding arrangements can for example be protrusions or recesses that provide a binary code telling the digital module what size the mask is.

In another embodiment the arrangement for enabling the digital module to identify at least one specification of the mask can be an RFID (radio frequency identification) arrangement allowing the digital module to wirelessly decide at least one characteristic of a mask based on a tag embedded in the mask.

In another embodiment the arrangement for enabling the digital module to identify at least one specification of the mask can be a magnetic arrangement allowing magnetic engagement at different spots in order to indicate at least one characteristic of the mask.

In one embodiment of the resuscitation arrangement the identification arrangement is at least one micro-switch or radio frequency identification tag.

In one embodiment of the resuscitation arrangement the digital module comprises a wireless communication arrangement adapted to communicate with at least one digital unit, preferably a main station.

In order to display more detailed information the digital module further comprises a wireless communication arrangement which in one embodiment is adapted to communicate with at least one digital unit. The digital unit can in one embodiment be similar to the digital module, however in another embodiment the digital unit is a base station working as a main unit for the resuscitation arrangement. In yet another embodiment the digital module can be for example a tablet or a smartphone with software adapted for the purpose.

In one embodiment of the resuscitation arrangement both the digital module and the digital unit are adapted to indicate low battery level and connection status between said digital module and said digital unit.

It is important that the information provided by the resuscitation arrangement is accurate at all times and that the operator immediately is notified if the information for some reason will cease or be non-trustworthy. Thereby, in one embodiment both the digital module and digital unit are adapted to indicate low battery level providing a warning to the operator that the digital module soon will cease from indicating and conducting measurements. Such an indication can for example be done with a pulsating illumination of the mask illumination arrangement, by a sound, or by any other form of indication that would catch the attention of the operator.

The illumination arrangement adapted to illuminate the mask can thereby be used for multiple purposes through different indications that can be differentiated by the operator. For example can different illumination colors be used and/or a pulse effect such as flashing the illumination arrangement.

In one embodiment of the resuscitation arrangement the digital module comprises an arrangement for storing data, preferably data gathered by the active sensor component.

For some application areas it is beneficial to store the data gathered by the digital module. Such information can in one embodiment be stored directly in the digital module and at a later stage be transferred to a computer, digital unit, or any other form of unit where it can be stored or analyzed. In another embodiment, all the information is directly transferred wirelessly to for example a digital unit wherein the information is stored.

In one embodiment of the resuscitation arrangement said digital module comprises a display.

In one embodiment the digital module also comprises a display that can present more detailed values from the measurements.

In one embodiment of the resuscitation arrangement the mask comprises a sealing arrangement arranged in at least one outlet adapted to engage with the digital module, wherein the sealing arrangement is broken when the digital module is arranged in engagement in said mask allowing a fluid connection between said mask and the digital module.

In one embodiment wherein the mask is a reusable mask arranged in a resuscitation arrangement the mask is airtight and is adopted to be usable without the digital module. However, when the digital module is arranged in engagement with the mask in order to monitor parameters a sealing engagement is broken by the digital module allowing a fluid connection between the passive and active sensor parts.

According to another aspect in a resuscitation arrangement for resuscitation of humans, preferably infants, said arrangement for resuscitation comprises a mask adapted to fit over the nose and mouth of said human, monitoring arrangements, and a digital module, wherein the monitoring arrangement comprises at least one passive sensor component and an active sensor component and performing the steps of:
detachably arrange said digital module comprising the active sensor component as a part of said mask comprising the passive sensor component,
establish a fluid connection between said passive sensor component and said active sensor component, and
monitor at least one parameter out of flow, leakage, volume, and pressure within said mask.

In one embodiment in a resuscitation arrangement the digital module is detachably arranged in a recess of said mask.

In one embodiment in a resuscitation arrangement said digital module is a reusable module and said mask is a disposable mask.

In one embodiment in a resuscitation arrangement said digital module further comprises an illumination arrangement to illuminate said mask and the further comprises the steps of:
determining if a value from the active sensor component is within a predetermined interval, and
if the value from the active sensor component is outside of said predetermined interval, illuminate said mask.

The solution furthermore provides additional benefits over prior art solutions, for example, in the education systems that previously exists there are multiple parts that shall be attached together in order to create a working resuscitation arrangement. Attaching multiple different hoses and adapters with different diameters is difficult when not under stress but next to impossible in situations that is life threatening to the patient. Furthermore, once assembled such solutions has proven to easily come apart during usage.

### BRIEF DESCRIPTION OF DRAWINGS

The solution is now described, by way of example, with reference to the accompanying drawings, in which:

FIG. 1 illustrates an isometric view of a preferred embodiment of the resuscitation arrangement comprising a mask and a digital module, wherein the digital module is arranged in a recess in said mask.

FIG. 2 illustrates an isometric view of a second embodiment of the resuscitation arrangement comprising mask and the digital module, wherein the digital module is arranged detachably to said mask.

FIG. 3 illustrates an isometric view of the digital module in further detail.

FIG. 4 illustrates an isometric view of the digital module in further detail according to another embodiment.

FIG. 5 illustrates a view of the base surface of the digital module according to one embodiment.

FIG. 6 illustrates a cross section view of the mask according to one embodiment of the resuscitation arrangement.

FIG. 7 illustrates a cross section view of the mask according to another embodiment of the resuscitation arrangement comprising means for illumination.

FIG. 8 illustrates one embodiment of the resuscitation arrangement for stationary use comprising a mask, a display, and a main unit.

FIG. 9 illustrates a schematic view of the components of a digital module in one embodiment of the resuscitation arrangement.

### DESCRIPTION OF EMBODIMENTS

In the following, a detailed description of the different embodiments of the invention is disclosed under reference to the accompanying drawings. All examples herein should be seen as part of the general description and are therefore possible to combine in any way in general terms. Individual features of the various embodiments and aspects may be combined or exchanged unless such combination or exchange is clearly contradictory to the overall function of the resuscitation arrangement.

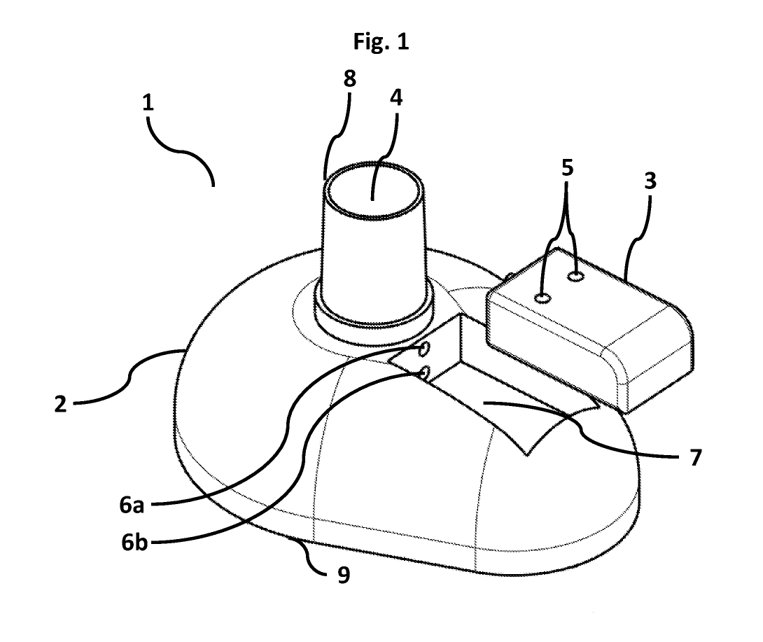

FIG. 1 illustrates an embodiment of the resuscitation arrangement 1 comprising a mask 2 adapted to fit over the nose and mouth of a human, a digital module 3, a recess 7 adapted to receive said digital module 3, and a tube 8 extending from said mask and adapted to be connected to a manual ventilator (not shown), a gas bottle (not shown), or any other means for supplying gas to the mask 7. The connection of tubing to the tube 8 creates a fluid connection making the inside 4 of the tube 8 the flow path 4 for said gas.

The mask 2 of the resuscitation arrangement 1 further comprise an open side 9 which in one preferred embodiment is the side adapted to fit over the nose and mouth of a human. The mask 2 is substantially hollow and while in engagement against a human face a fluid connection is established between said nose and mouth of the human and the flow path 4.

The digital module 3 can further comprise one or more indication arrangements 5, such as LEDs arranged on the top side of said digital module 3 in order to indicate for example if the digital module 3 is fully charged and/or active. The person skilled in the art understands that any number of LEDs can be arranged on the top side of the digital module 3 within the scope of the present solution.

The mask 2 further comprises means 6a, 6b for connection between the active and passive sensor component preferably arranged in said recess 7. The means 6a, 6b for connection between the active and passive sensor component can for example be apertures 6a, 6b or extending piping portions 6a, 6b that are adapted to fit in apertures in the digital module 3.

It is understood that the resuscitation arrangement may comprise many different additional parts such as a manual ventilator, a gas tube, connection tubes, a main unit, a display, or any other parts relevant to the resuscitation arrangement. In order to further clarify the manual ventilator mentioned at numerous occasions above can for example be a BVM or T-piece. It may also be solutions comprising a flow-inflating or self-inflating bag.

In one embodiment, prior of starting the resuscitation process a care provider or an assistant to a care provider removes the digital module 3 from a charger, or any other form of storage, and place the digital module 3 in engagement with the mask 2, preferably within a recess 7. The care provider checks that the digital module 3 is active by looking at the indication arrangements 5 and connects a gas source, such as a gas bottle or a manual ventilator, to the tube 8 creating a fluid connection between the gas sources and the mask 2 through the flow path 4. The mask 2 is placed on the patient with the open side 9 towards the patient's face covering the nose and mouth. The resuscitation process is started.

During the resuscitation process the digital module 3 monitors at least one parameter chosen from flow, volume, leakage, and pressure within the mask 2 through the means 6a, 6b for connection between the active and passive sensor components. The at least one active sensor component is arranged within the digital module 3 while the at least one passive sensor component is arranged within said mask 2 or more specifically said flow path 4.

The care provider constantly receives feedback relating from the process through for example illumination of the mask 2, sounds, or by looking at an external display. The feedback could further in one embodiment be for example tactile feedback, vibrations, sounds, or any form of other feedback adapted to attract the care providers attention.

FIG. 1 further illustrates how the digital module 3 is attached to the mask 2 in the resuscitation arrangement. In one embodiment the digital module 3 is attached to the mask 2 through means of magnetic engagement. This is done through that the mask 2 comprises small pieces, or alternatively one larger piece, of magnetic material that is molded in to the mask 2. The digital module 3 comprises corresponding magnetic means, such as a permanent magnet, that is adapted to engage with the magnetic material in the mask 2 and thereby secure the digital module 2 to the mask. In one embodiment, the magnetic means are reversed placing the permanent magnet within the mask.

In another embodiment the digital module 3 is instead secured by mechanical fastening means, such as a clip.

In yet another embodiment the digital module 3 is secured by a tight fit between the mask, made out of for example silicon, and the digital module.

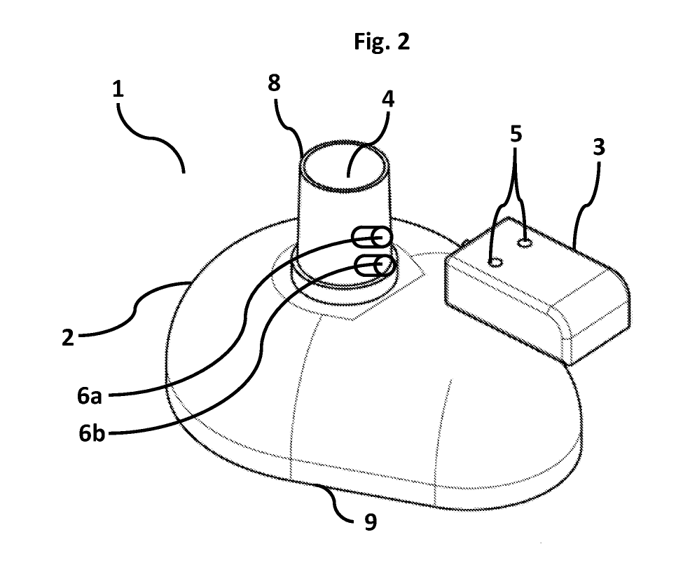

FIG. 2 illustrates another embodiment of the resuscitation arrangement 1 wherein the digital module 3 is adapted to be attached to the mask 2 without any corresponding recess. The mask comprises means for connection between the active and passive sensor components 6a, 6b that for example can be arranged on the outer peripheral of the tube 8.

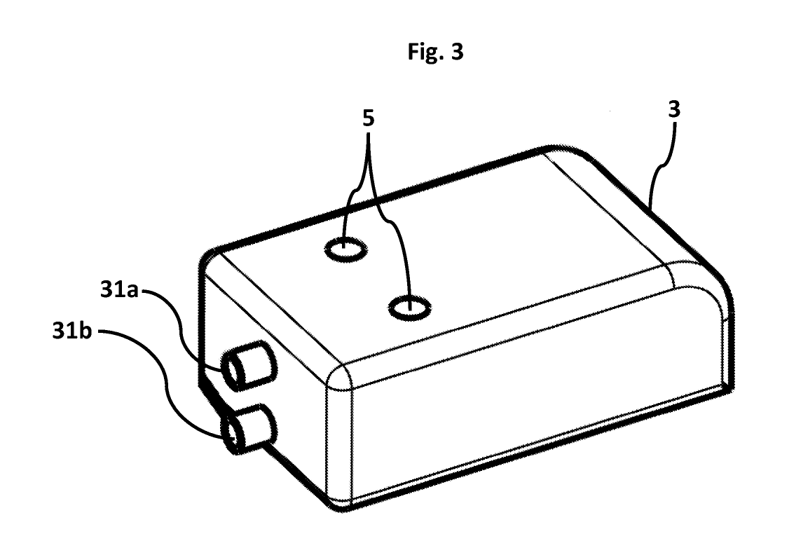

FIG. 3 illustrates the digital module 3 in detail comprising the indication arrangements 5 and corresponding means 31a, 31b for connection between the active and passive sensor components adapted to engage into fluid connection with the means 6a, 6b for connection between the active and passive sensor components arranged on the mask 2.

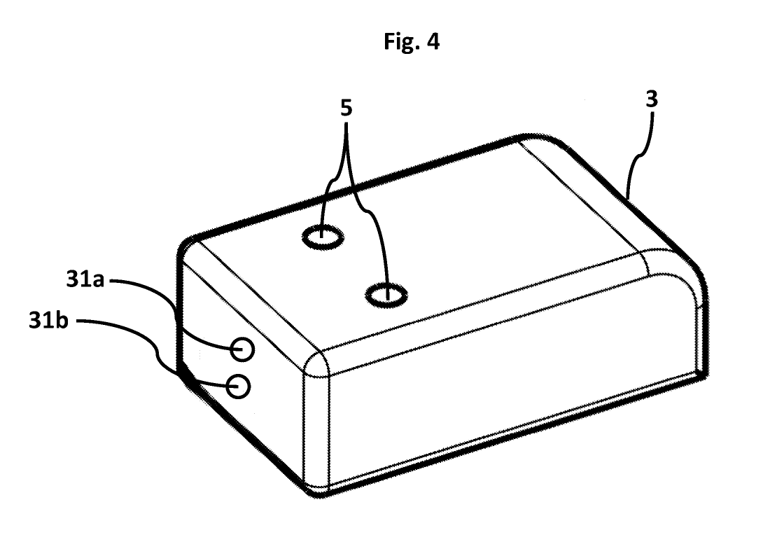

FIG. 4 illustrates another embodiment of the digital module 3 comprising inverted means 31a, 31b for connection between the active and passive sensor components. Those means 31a, 31b are still adapted to engage with the means 6a, 6b for connection between the active and passive sensor components arranged on the mask 2 but the female and male adapters have switched places. This is a preferred embodiment and beneficial due to that no external parts are introduced to the flow path 4 of the mask 2 which decreases the risks of contaminating the gas within the mask 2.

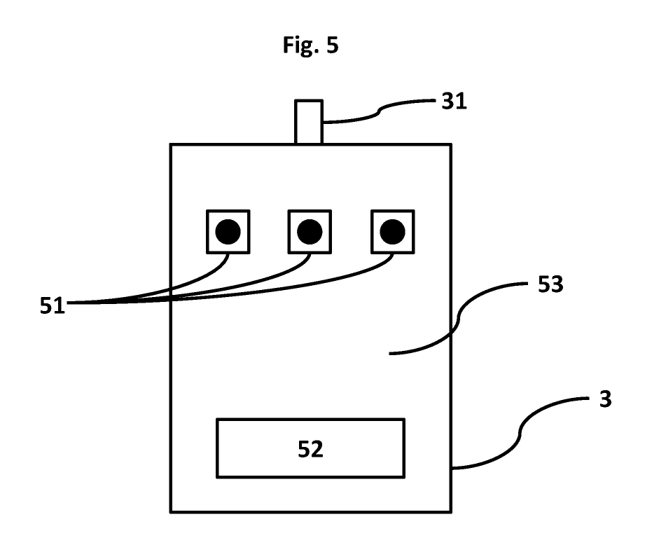

FIG. 5 illustrates the digital module 3 from a view where the base surface 53 that normally is oriented downwards towards the mask 2 during usage is shown. In one embodiment at least one identification arrangement 51 enabling the digital module 3 to identify at least one specification of the mask 2 is arranged on or in the close vicinity of the base surface 53. For example, in a preferred embodiment multiple micro-switches 53 are arranged to engage with corresponding recess or protrusions in the mask 2 providing information such as the mask size, the mask type, the type of passive sensor components arranged in the mask, the amount of dead space in the mask, or any other information that is useful for the digital module 2. The information could for example be in the form of a binary code generated from micro-switches 51 that are currently actuated. For example, if three micro-switches are arranged on the base surface 53, in one embodiment the mask 2 has two protrusions corresponding to the first and last micro-switch 51 generating a binary code of “101” corresponding to for example a size of the mask 2 and what kind of passive sensor components that are arranged within the mask.

In another embodiment the identification arrangement 51 are any other form of identification arrangement 51 such as magnetic actuators, an RFID tag, or any other form of identification arrangement 51.

FIG. 5 further illustrates an illumination arrangement 52 adapted to illuminate said mask. The illumination arrangement 52 illuminates the mask by for example sending a light generated by a light source in the digital module 3 into the mask 2. The mask thereby illuminates through for example an illuminative material or through fiber optics arranged within the mask 2.

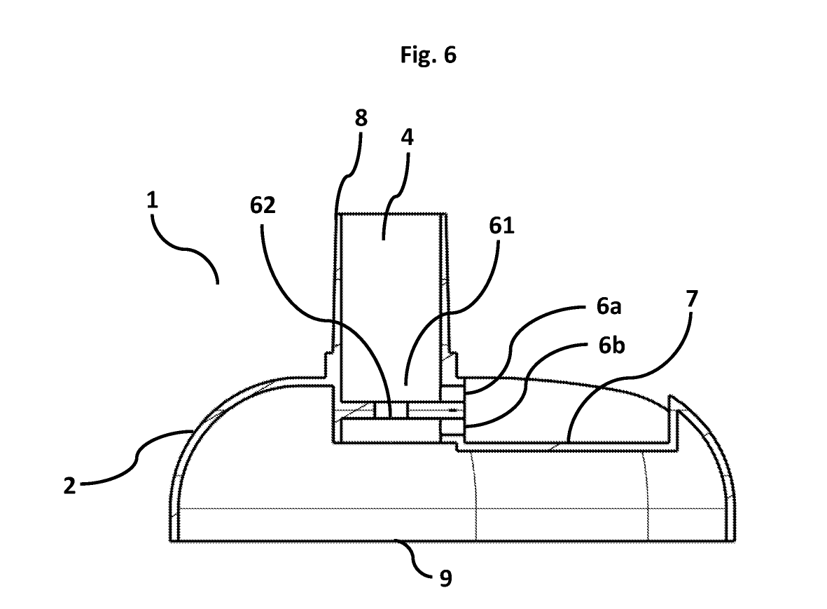

FIG. 6 illustrates a cross section of the mask 2 illustrating the recess 7 and the passive sensor component 61 in further detail. In the embodiment illustrated in FIG. 6 the mask 2 comprises one passive sensor component 61 arranged within the flow path 4. The means 6a, 6b for connection between the active and passive sensor component are working as outlets 6a, 6b allowing for example a pressure to be measured by an active component within the digital module 3.

The passive sensor component 61 comprises a constriction 62 arranged between the outlet 6a and the outlet 6b creating a pressure difference between the two outlets 6a, 6b that can be measured by an active sensor component within the digital module 3. The constriction 62 can be any form of constriction 62 ranging from a narrow hole to a more complex geometry comprising flexing details. For example, in one embodiment a thin plastic layer is applied as a constriction 62. The thin plastic layer comprises a cut that allows the thin layer to flex in any direction allowing air to pass through a part of the area. However, the person skilled in the art understands that any form of constriction 62 can be used.

In another embodiment the passive sensor component 61 is another type of sensor.

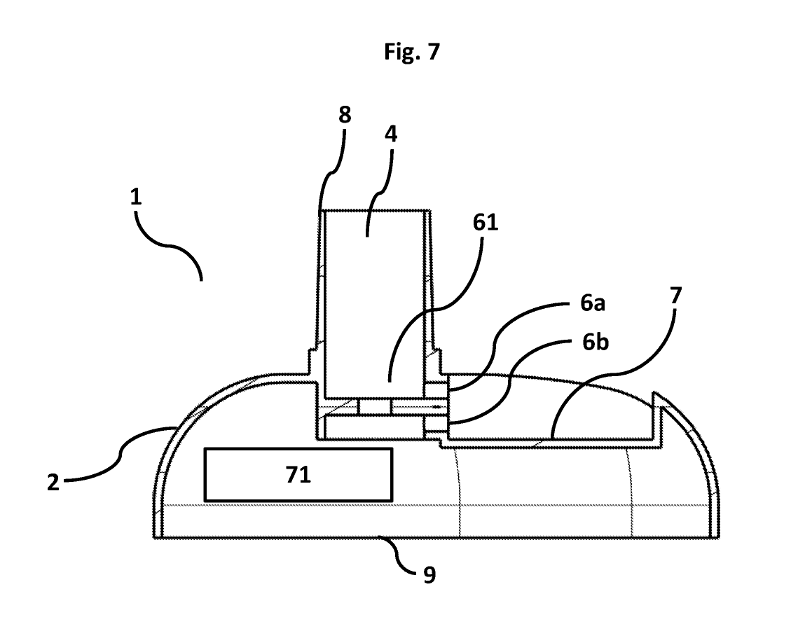

FIG. 7 illustrates another embodiment wherein the mask 2 further comprises an illumination arrangement 71 adapted to illuminate the mask 2. In one embodiment the illumination arrangement 71 comprises means to illuminate the mask, such as LEDs or any other form of illumination device. In another embodiment, the illumination arrangement 71 is arranged to distribute light produced by a light source located in the digital module 3. The illumination arrangement 71 can thereby for example be fiber optics arranged within the mask, electroluminescent materials, or light distribution materials such as light guiding plastic.

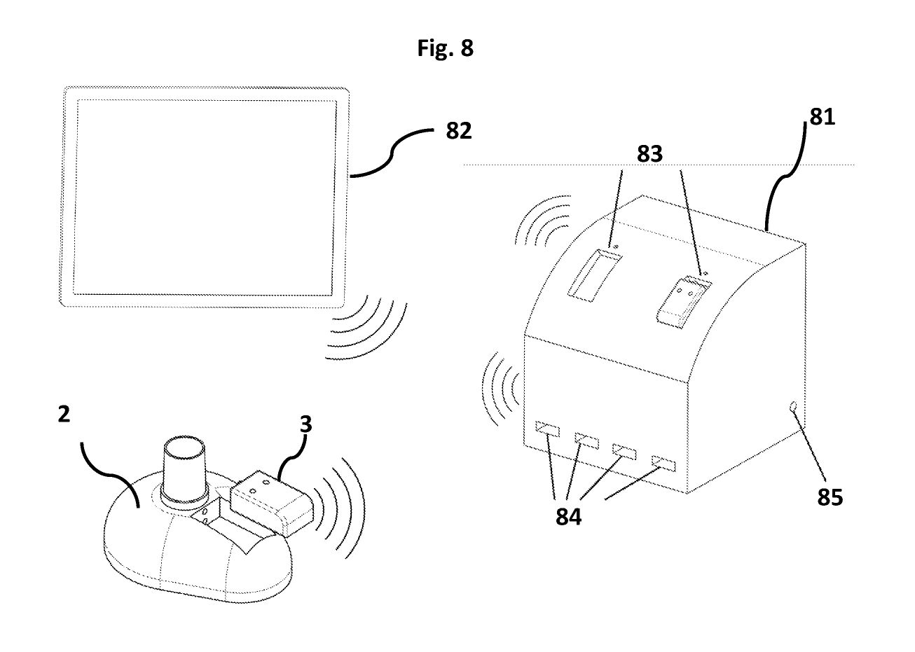

FIG. 8 illustrates one embodiment of the resuscitation arrangement for stationary use in for example a hospital comprising a main unit 81, a display 82, the digital module 3, and the mask 2. The main unit 81 can be any form of main unit 81 that is adapted to communicate with at least one digital module 3. The main unit 81 is in a preferred embodiment also a docking station for the digital module 3 providing the possibility to transfer additional data not sent via the wireless protocol, as well as comprising the possibility to charge the digital module 3. Charging and docking of the digital module 3 can in one embodiment be performed in a docking station 83. The main unit 81 could preferably further comprise a power inlet 85 for connection of said main unit 81 to the power grid, and connections 84 for additional displays. The connections 84 could for example be VGA, HDMI, DVI, or any other form of connection suitable to attach a display 82. In another embodiment all displays 82 are wireless, and in yet another embodiment the connections 84 are for connection of additional sensors, for example a heart rate sensor.

In one embodiment the display 82 is a mobile unit, such as a smartphone or tablet, which is adapted to display the information provided by the main unit 81 or the digital module 3.

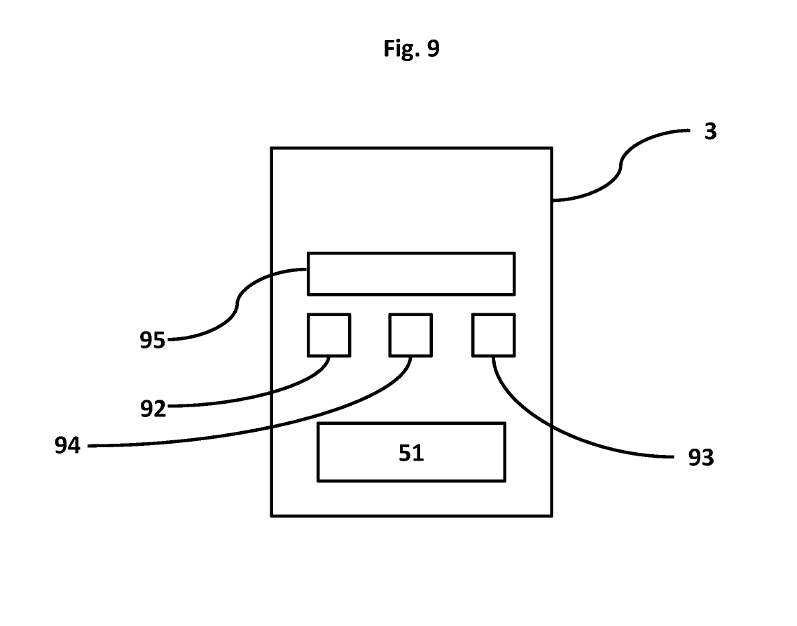

FIG. 9 illustrates a schematic view of the digital module 3 comprising at least a processor 92 or a CPU 92, a memory 94, at least one active sensor component 95 and wireless communication arrangement 93 adopted to communicate over at least one wireless network such as WiFi, Bluetooth, ZigBee, or any other form or wireless network or communication protocol.

In one embodiment the care provider, or any other qualified personnel, is able to enter information about the patient in order to get better feedback. For example, the weight is information that would improve the readings.

1. A resuscitation arrangement for resuscitation of humans, preferably infants, wherein said arrangement for resuscitation comprises a mask adapted to fit over the nose and mouth of said human, monitoring arrangements adapted to monitor at least one parameter chosen from flow, volume, leakage, and pressure within said mask and a digital module adapted to enable measurement of at least one of the parameters, wherein the monitoring arrangement comprises at least one passive sensor component and an active sensor component, wherein said digital module is adapted to be detachably arranged as a part of said mask, and wherein the passive sensor component is arranged within the mask and the active sensor component is arranged in the digital module.

2. The resuscitation arrangement according to claim 1, wherein
the digital module is adapted to be detachably arranged in a recess of the mask.

3. The resuscitation arrangement according to claim 1, wherein the digital module is a reusable module and the mask is a disposable mask.

4. The resuscitation arrangement according to claim 1, wherein the digital module is detachably arranged as part of said mask through a magnetic engagement.

5. The resuscitation arrangement according to claim 1, wherein the passive sensor component comprises a first and second sensor outlet adapted to be in fluid connection with the digital module, and wherein said passive sensor component comprises an arrangement creating a pressure difference between said first and second sensor outlet.

6. The resuscitation arrangement according to claim 1, wherein the digital module further comprises an illumination arrangement adapted to illuminate the mask.

7. The resuscitation arrangement according to claim 6, wherein
the illumination arrangement comprises at least one light guiding material, preferably fibre optics or light guiding plastic.

8. The resuscitation arrangement according to claim 1, wherein the mask comprises an identification arrangement enabling for said digital module to identify at least one specification of said mask.

9. The resuscitation arrangement according to claim 8, wherein the identification arrangement is at least one of a micro-switch or a radio frequency identification tag.

10. The resuscitation arrangement according to claim 1, wherein the digital module comprises a wireless communication arrangement adapted to communicate with at least one digital unit, preferably a main station.

11. The resuscitation arrangement according to claim 10, wherein both the digital module and the digital unit are adapted to indicate low battery level and connection status between said digital module and digital unit.

12. The resuscitation arrangement according to claim 1, wherein the digital module comprises an arrangement for storing data, preferably data gathered by the active sensor component.

13. The resuscitation arrangement according to claim 1, wherein said digital module comprises a display.

14. The resuscitation arrangement according to claim 3, wherein the mask comprises a sealing arrangement arranged in at least one outlet adapted to engage with the digital module, wherein the sealing arrangement is broken when the digital module is arranged in engagement in said mask allowing a fluid connection between said mask and the digital module.

15. A method in an arrangement for resuscitation of humans, preferably infants, wherein said arrangement for resuscitation comprises a mask adapted to fit over the nose and mouth of said human, monitoring arrangements, and a digital module, wherein the monitoring arrangement includes at least one passive sensor component and an active sensor component, wherein the digital module including the active sensor component as a part of said mask having the passive sensor component, wherein the method comprises the steps of:
establishing a fluid connection between said passive sensor component and said active sensor component, and
monitoring, by the digital module, at least one parameter out of flow, leakage, volume, and pressure within said mask.

16. The method according to claim 15, wherein the digital module is detachably arranged in a recess of said mask.

17. The method according to claim 15, wherein said digital module is a reusable module and said mask is a disposable mask.

18. The method according to claim 15, wherein said digital module further includes an illumination arrangement to illuminate said mask and the method further comprises the steps of:
determining if a value from the active sensor component is within a predetermined interval, and
if the value from the active sensor component is outside of said predetermined interval, illuminating said mask.
